(19) United States
(12) Patent Application Publication
(10) Pub. No.: US 2024/0264169 A1
(43) Pub. Date: Aug. 8, 2024

(54) METHOD FOR THE QUANTIFICATION OF THE ACTIVATION STATE OF G-PROTEIN-COUPLED RECEPTORS IN A HUMAN BIOLOGICAL SAMPLE

(71) Applicant: ALDA S.R.L., Napoli (IT)

(72) Inventors: Alberto LUINI, Napoli (IT); Matteo LO MONTE, Napoli (IT); Vincenzo Manuel MARZULLO, Napoli (IT); Federica LICCARDO, Napoli (IT)

(73) Assignee: ALDA S.R.L., Napoli (IT)

(21) Appl. No.: 18/564,054

(22) PCT Filed: May 25, 2022
(86) PCT No.: PCT/IB2022/054888

§ 371 Date: Nov. 24, 2023

(30) Foreign Application Priority Data
May 27, 2021 (IT) 102021000013928

(51) Int. Cl. G01N 33/68; G01N 33/542

(52) U.S. Cl. CPC G01N 33/6863; G01N 33/542; G01N 2333/5421; G01N 2333/726

(57) ABSTRACT

A method for measurement of an activation state of G-protein-coupled receptors comprises: supplying a human biological sample; preparing a fluorescent donor probe comprising an anti-GPCR Fab fragment or antibody and a fluorophore group linked via amide bond to an amino group of the N-terminal amino acid of the light chain and/or of the N-terminal amino acid of the heavy chain of the Fab fragment or antibody; preparing a fluorescent acceptor probe comprising an anti-G-protein and/or anti-arrestin Fab fragment or antibody and a fluorophore group linked via amide bond to an amino group of the N-terminal amino acid of the light chain and/or of the N-terminal amino acid of the heavy chain of the Fab fragment or antibody; setting the human biological sample in contact with the fluorescent acceptor probe and with the fluorescent donor probe; sensing the activation state of the GCPR receptors by means of fluorescence microscopy techniques.

### TECHNICAL FIELD

The present invention relates to a method for the quantification of the activation state of G-protein-coupled receptors in a human biological sample.

### BACKGROUND ART

As is well known, the super-family of G protein-coupled receptors (GPCR) represents the largest class of receptors in the mammalian cell membrane. These receptors drive numerous signaling pathways involved in the regulation of a wide range of physiological and pathological processes. GPCRs, in fact, are molecules capable of selecting the activation of specific signals and thus generating well-structured responses. Frequently, the activation status of these receptors correlates with a condition of diagnostic, prognostic, or treatment-response interest. As such, they are considered effective targets for drug development. Already, an estimated 30-50% of drugs used in the clinic affect the functions of the GPCR family members.

These receptors and their activation state are involved in numerous pathologies. For this reason, there is a particular need for an analytical tool that can reliably, precisely and accurately determine their activation status.

Although stimulation of GPCRs can modulate a wide variety of separate signaling pathways, only two are mainly of particular interest: the first is characterized by G protein-coupled activation without β-arrestin recruitment and, the second involves recruitment of arrestin-2 by the receptor.

In detail, interleukin-8 (IL-8 or neutrophil chemotactic factor) is a proinflammatory chemokine that induces chemotaxis and promotes phagocytosis in target cells.

Interleukin-8 is often associated with inflammation; because its interleukin-8 secretion is increased by oxidative stress, which in turn causes recruitment of inflammatory cells to cause a further increase in oxidative stress mediators, it makes it a key parameter in localized inflammation.

Chronic inflammatory diseases (such as e.g., colitis, hepatitis, pancreatitis etc.) are characterized by an increased risk of developing cancer in the affected organs. In particular, the substances released into the tissue site of inflammatory processes, reduce the activity of the most potent tumor suppressor, the p53 protein, normally present in cells, thus causing the cells in the tissue to acquire the characteristics of neoplastic cells. Along with these specific characteristics, IL-8 is also known to be a potent promoter of angiogenesis and invasiveness in cancer.

Increased expression of IL-8 and/or its receptors has been characterized in diverse tumor cells, endothelial cells, infiltrating neutrophils and tumor-associated macrophages, thus suggesting that IL-8 may function as a significant regulatory factor within the tumor microenvironment. As a consequence of the diversity of effectors and downstream molecular targets, IL-8 signaling promotes angiogenic responses in endothelial cells, enhances proliferation and survival of endothelial and tumor cells and potentiates the migration of tumor cells, endothelial cells and neutrophil infiltration into the tumor-involved region. Accordingly, IL-8 expression correlates with angiogenesis, tumorigenicity and tumor metastasis in numerous in vivo xenograft and orthotopic models. Recently, IL-8 signaling has been implicated in the regulation of androgen receptor transcriptional activity, supporting the transition to androgen-independent proliferation of prostate cancer cells. In addition, stress- and drug-induced IL-8 signaling has been shown to confer chemotherapeutic resistance in cancer cells.

Critical phenomena underlying metastatic tumor evolution also involve many chemokines and cytokines (mainly interleukins), among them IL-8 plays a prominent role in influencing epithelial-to-mesenchymal transition (EMT), facilitating detachment of tumor cells from the primary tumor mass, regulating cell migration, promoting seeding by means of circulating tumor cells (CTC) and stimulating proliferation.

Generally, it is possible to determine the concentration of cytokines in the bloodstream of patients using standard analytical methods, such as e.g. ELISA tests or mass spectrometry. However, these measurements, it is believed, have not so far at least revealed clear prognostic or diagnostic significance. Indeed, plasma concentrations of both pro-inflammatory and anti-inflammatory cytokines tend to change very easily and prove to fluctuate widely in many immune-mediated diseases, such as rheumatoid arthritis. Moreover, the activation of cytokine receptors involved in inflammatory processes is often not directly correlated with the level of cytokine expression, either because the receptor response may depend on regulatory factors independent of the level of ligand, or because the receptors themselves may be subject to “Single Nucleotide Polymorphisms” (SNPS) or may simply be expressed in variable amounts. All these factors make the receptor response following interaction with cytokines variable and unpredictable. To date, there is still no method available to effectively measure the activation level of IL-8 receptor.

### DESCRIPTION OF THE INVENTION

The main aim of the present invention is to devise a method for the quantification of the activation state of G-protein-coupled receptors in a whole biological sample, i.e., by preventing the latter from undergoing treatments aimed at removing one or more of its components.

Another object of the present invention is to devise a method for the quantification of the activation state of G-protein-coupled receptors which will enable the recognition and measurement of protein interactions characterizing the activation state of G-protein-coupled receptors themselves and correlate the latter to pathological states.

Another object of the present invention is to devise a method for the quantification of the activation state of G-protein-coupled receptors that can overcome the aforementioned drawbacks of the prior art within the framework of a simple, rational, easy-to-use and cost-effective solution.

The aforementioned objects are achieved by the present method for the quantification of the activation state of G-protein-coupled receptors having the characteristics of claim 1.

### BRIEF DESCRIPTION OF THE DRAWINGS

Other characteristics and advantages of the present invention will become more apparent from the description of a preferred, but not exclusive, embodiment of a method for the quantification of the activation state of G-protein-coupled receptors, illustrated by way of an indicative, yet non-limiting example, in the accompanying tables of drawings in which:

FIG. 1a is an illustration indicative of the localization and expression levels of the tagged CXCR1 protein (expression of CXCR1 (clone #501) in L1.2);

FIG. 1b is an illustration indicative of the localization and expression levels of CXCR1 protein (clone #501) in A-375 melanocytes);

FIGS. 2a-c are illustrations indicative of the validation of the following ANTIBODIES and FAB pairs in human blood for monoclonal #501. In detail, the following antibody-only pairs were validated in human blood for antiserum PA5-87555 and PA5-35089 directed against β-Arrestin1, and antiserum PA1-1000 directed against G-α-I protein isoforms 1 and 2:
receptor//G-α-I (1+2)
receptor//β-Arr1(ARRESTIN-1)
receptor//β-Arr2(ARRESTIN-2);

FIG. 2a shows the expression and localization of endogenous CXCR1 protein visualized by means of antibody clone #501. FIG. 2b shows the expression of endogenous β-Arrestin2 protein labeled by monoclonal antibody clone 5E12.1.

FIG. 2c is representative of the merge of the two channels. Cells: human neutrophils from a healthy donor, fixed in the quiescent state.

FIGS. 3a-3b are images related to the expression and localization of endogenous CXCR1 protein, visualized with the Fab obtained from monoclonal antibody #3HCLC. Cells: human neutrophils from a healthy donor fixed at 10 min after IL-8 (100 nM) stimulus. Localization of CXCR1 protein in intracellular vesicles (probably endosomes) is particularly evident;

FIG. 3c is a representative image of the expression and localization of endogenous G-α-I protein, visualized with the polyclonal antibody. Cells: human neutrophils from a healthy donor fixed in HANS buffer after gradient purification and lysis of erythrocytes;

FIG. 4 shows: a) diagram of time blocks used for experiments in the primary human neutrophil line; b) modulation of FRET signal in samples subjected to different conditions: CTRL=control, A: treatment with IL-8 (100 nM, 10 minutes), B1: treatment with competitive inhibitor, B2=combined treatment with competitive inhibitor+IL-8 (100 nM, 10 minutes), B3=combined treatment with competitive inhibitor+allosteric Navarixin inhibitor (10 uM, 10 minutes); c) morphology of cells activated by interleukin-8 stimulus (100 nM, 10 minutes); d) distribution of FRET values recorded in a sample of cells fixed in the quiescent state (blue) and in a sample fixed during the activation state (IL-8 100 nM, 10 minutes): the statistical significance related to the difference in the values found in the two different conditions (treated sample and quiescent sample) is high and allows defining two discrete FRET ranges for the population of activated cells, and the population of non-activated cells, respectively;

FIG. 5 shows receptor-navarixin or receptor-reparixin interaction. The graph on the left shows the results obtained with the allosteric Navarixin modulator used at the concentration of 10 uM (not shown in graph), the graph on the right shows the results obtained with the allosteric REPARIXIN modulator, at the concentration of 40 ug/mL (corresponding to 92 uM) and 100 ug/mL (corresponding to 230 uM);

FIG. 6 shows receptor-gi2 interaction—FRET-FLIM analysis of the CXCR1-GaI interaction pair. Control system, activation with IL-8 (10 nM), donor-only (anti-CXCR1), IL-8-activated system in the presence of Navarixin 100 μM) were compared;

FIG. 7 shows the FRET assay for the measurement of CXCR1//Arrβ interaction on neutrophils, operating in triplicate;

FIG. 8 shows a graph regarding the decay of donor probe fluorescent emission under the three different conditions: CTRL (control, corresponding to quiescent cells), Donor Only (internal negative control in which the acceptor probe is missing), Reparixin 100 micrograms/mL, which corresponds to the highest tested assay.

### EMBODIMENTS OF THE INVENTION

The method for the quantification of the activation state of GPCR receptors, comprises at least the following phases:
supplying a human biological sample;
preparing at least one fluorescent donor probe comprising an anti-GPCR Fab fragment or antibody and a fluorophore group linked via amide bond to an amino group of the N-terminal amino acid of the light chain and/or of the N-terminal amino acid of the heavy chain of said Fab fragment or antibody;
preparing at least one fluorescent acceptor probe comprising an anti-G-protein and/or anti-arrestin Fab fragment or antibody and a fluorophore group linked via amide bond to an amino group of the N-terminal amino acid of the light chain and/or of the N-terminal amino acid of the heavy chain of the Fab fragment or antibody;
setting the human biological sample in contact with the fluorescent acceptor probe and with the fluorescent donor probe;
sensing the activation state of the GCPR receptors by means of fluorescence microscopy techniques, preferably super-resolution confocal fluorescence microscopy.

It is specified that within the scope of this disclosure the expression “human biological sample” refers to any sample obtained from human biological tissues and fluids such as blood, saliva, urine and the cells, comprising all molecular fractions (proteins, RNA, DNA, etc.) derivable from them, originating from healthy individuals or those with disease.

Preferably, the human biological sample is blood.

In more detail, the above biological sample is obtained from a patient treated with a pharmacological modulator of interleukin-8 receptors.

It is specified that within the scope of this disclosure, by the term “pharmacological modulator of interleukin-8 receptors” we mean any drug therapy with direct or indirect effects on the interleukin-8-mediated cell signaling pathway.

Advantageously, the donor fluorescent probe comprises an anti-CXCR1 and anti-CXCR2 Fab fragment or antibody.

Preferably, the fluorescent acceptor probe comprises an anti-subunity G-α-I and/or anti-ß-arrestin 1 and/or anti-ß-arrestin 2 Fab fragment or antibody.

In detail, in the Fab fragment or antibody according to the invention, the amino group of the N-terminal amino acid of the light chain and/or of the N-terminal amino acid of the heavy chain is linked via amide bond to a group Z of formula:

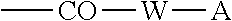

wherein:
A is a fluorophore group,
W is chosen from C₁-C₁₀ alkyl, —CH₂CH₂(CH₂CH₂O)ₘCH₂CH₂—X—, or —CH₂—CH₂OCH₂CH₂—Z—Y—CH₂CH₂OCH₂CH₂—X—,
wherein m is between 0 and 8, X is chosen from CO and NH, Y is chosen from CH₂CH₂OH, pyrimidine, methoxyphenyl, or a compound of formula

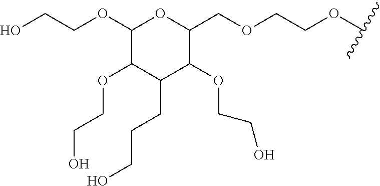 [SMILES: *OCCOCC1OC(OCCO)C(OCCO)C(CCCO)C1OCCO]

Preferably, in the above antibody or Fab, the fluorophore group A is chosen from the following groups:
fluorophore CF568
fluorophores derived from xanthene or carbocyanins, preferably of formula:

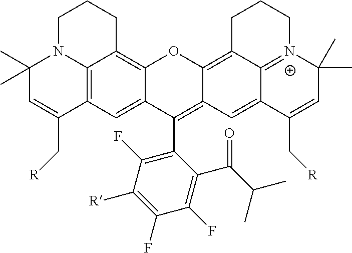

wherein R is a group chosen from —OPO₃H₂ and —OH and R′ is chosen from —OSO₃— and —F;
2-[(1E,3E)-5-[(2Z,3S)-3-(5-methoxy-5-oxopentyl)-3-methyl-5-sulfo-1-(3-sulfopropyl)-2,3-dihydro-1H-indol-2-ylidene]penta-1,3-dien-1-yl]-3,3-dimeth yl-5-sulfo-1-(3-sulfopropyl)-3H-indol-2-yl (AF647), having structure

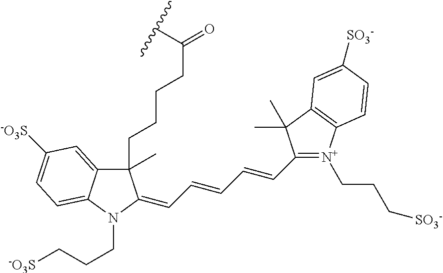

2-(2,2,10,10-tetramethyl-4,8-bis(sulfonatomethyl)-2,10-dihydro-1H-pyrano[3,2-g:5,6-g′]diquinolin-11-ium-6-yl)terephthalate (AF568), both regioisomers, having structure:

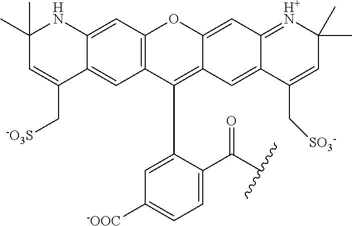

e
2-[(1E,3Z)—

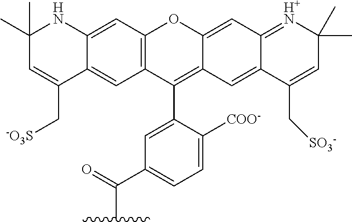

3-[3-(3-carbamoylpropyl)phenyl]-5-[(2Z)-3,3-dimethyl-5-sulfonato-1-(3-sulfonatopropyl)-2,3-dihydro-1H-indol-2-ylidene]penta-1,3-dien-1-yl]-5-chloro-3,3-dimethyl-7-(3-sulfonatopropyl)-3H-pyrrolo[2,3-b]pyridin-7-ium;
2-[(E)-2-[(3E)-3-{2-[(2Z)-1-(5-carbamoylpentyl)-3,3-dimethyl-5-sulfonato-2,3-dihydro-1H-indol-2-ylidene]ethylidene}-2-(4-sulfonatophenoxy)cyclohex-1-en-1-yl]ethenyl]-3,3-dimethyl-1-(4-sulfonatobutyl)-3H-indol-1-ium-5-sulfonate;
(1P)-2-(6-amino-3-iminiumyl-4,5-disulfonato-3H-xanthen-9-yl)-4-carbamoylbenzoate;
1-(5-carbamoylpentyl)-3,3-dimethyl-2-[(1E,3E)-5-[(2Z)-1,3,3-trimethyl-2,3-dihydro-1H-indol-2-ylidene]penta-1,3-dien-1-yl]-3H-indol-1-ium;
2-[(1E,3E)-5-[(2Z)-1-(5-carbamoylpentyl)-3,3-dimethyl-5-sulfonato-2,3-dihydro-1H-indol-2-ylidene]-3-methylpenta-1,3-dien-1-yl]-3,3-dimethyl-1-(4-sulfonatobutyl)-3H-indol-1-ium-5-sulfonate;
2-[(1E,3E)-5-[(1R,2Z)-1-(5-carbamoylpentyl)-3,3-dimethyl-5-sulfonato-2,3-dihydro-1H-indol-1-ium-2-ylidene]penta-1,3-dien-1-yl]-3,3-dimethyl-1-(4-sulfonatobutyl)-3H-indol-1-ium-5-sulfonate;
2-tert-butyl-4-[(1E)-3-[(2E)-1-(5-carbamoylpentyl)-3,3-dimethyl-5-sulfonato-2,3-dihydro-1H-indol-2-ylidene]prop-1-en-1-yl]-9-ethyl-8,8-dimethyl-8H,9H-1,4-chromeno[7,6-b]pyridin-1-ylium;
4-[(1E)-3-[(2Z,3S)-3-(5-carbamoylpentyl)-3-methyl-6-sulfonato-1-(3-4)-2,3-dihydro-1H-indol-2-ylidene]prop-1-en-1-yl]-8,8-dimethyl-2-phenyl-6-(sulfonatomethyl)-9-(3-sulfonatopropyl)-8H,9H-1λ4-chromeno[7,6-b]pyridin-1-ylium;
2-tert-butyl-4-[(1E,3E)-5-[(2E)-1-(5-carbamoylpentyl)-3,3-dimethyl-5-sulfonato-2,3-dihydro-1H-indol-2-ylidene]penta-1,3-dien-1-yl]-7-(diethylamino)-1λ4-chromen-1-ylium;
N,N-dimethyl-4-[(2E)-1,5,5-tris[4-(dimethylamino)phenyl]penta-2,4-dien-1-ylidene]cyclohexa-2,5-dien-1-iminium;
2-[(1E,3E)-5-[(2Z)-1-(5-carbamoylpentyl)-3,3-dimethyl-5-sulfonato-2,3-dihydro-1H-indol-2-ylidene]penta-1,3-dien-1-yl]-3,3-dimethyl-1-(4-sulfonatobutyl)-3H-indol-1-ium;
28R)-16-{2-[(3-carbamoylpropyl)(methyl)carbamoyl]phenyl}-3-oxa-9λ⁵,23-diazaheptacyclo[17.7.1.1⁵,⁹.0², ¹⁷.0⁴,¹⁵.0²³,²⁷.0¹³,²⁸] octacosa-1(27),2(17),4,9,13,15,18-heptaen-9-ylium;
2-[(1E,3E)-5-[(2Z)-1-(5-carbamoylpentyl)-3,3-dimethyl-2,3-dihydro-1H-indol-2-ylidene]penta-1,3-dien-1-yl]-1,3,3-trimethyl-3H-indol-1-ium;
2-[(1E,3E)-5-[(1Z,2R)-1-(5-carbamoylpentylidene)-3,3-dimethyl-2,3-dihydro-1H-1λ⁵-indol-1-ylium-2-yl]penta-1,3-dien-1-yl]-3,3-dimethyl-1-(4-sulfonatobutyl)-3H-indol-1-ium;
4-carbamoyl-2-[13-(dimethylamino)-5-(dimethyliminiumyl)-2,2-dimethyl-2-silatricyclo[8.4.0.0³,⁸]tetradeca-1(10),3,6,8,11,13-hexaen-9-yl]benzoate; e
2-[(1E,3E)-5-[(2Z)-1-(5-carbamoylpentyl)-3,3-dimethyl-5-sulfonato-2,3-dihydro-1H-indol-2-ylidene]penta-1,3-dien-1-yl]-3,3-dimethyl-1-(4-sulfonatobutyl)-3H-indol-1-ium;
(7S,17R)-12-[4-(methoxycarbonyl)phenyl]-7,8,8,16,16,17-hexamethyl-2-oxa-6,18-diazapentacyclo[11.7.0.^{03,11}.0^{5,9}.0^{5,19}]icosa-1(13),3,5,9,11,14,19-heptaen-6-ium-4,20-disulfonate (AF532), having structure

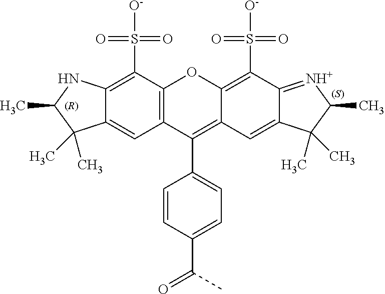

[10,10,22,22-tetramethyl-20-(sulfomethyl)-16-{2,3,4,5-tetrafluoro-6-[(4methoxy-4-oxobutyl)(methyl)carbamoyl]phenyl}-3-oxa-9lambda4,23-diazaheptacyclo[17.7.1.1^{5,9}.0^{2,17}.0^{4,15}.0^{23,27}.0^{13,28}]octacosa-1,4,9(28),11,13,15,17,19(27),20-nonaen-12-yl]methanesulfonic acid (ASred), having structure

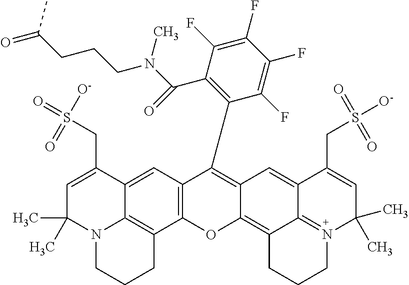

2-({3-[12,20-bis(hydroxymethyl)-10,10,22,22-tetramethyl-3-oxa-9lambda4,23-diazaheptacyclo[17.7.1.1^{5,9}.0^{2,17}.0^{4,15}.0^{23,27}.0^{13,28}]octacosa-1,4,9(28),11,13,15,17,19(27),20-onaen-16-yl]-2,5,6-trifluoro-4-[(4-methoxy-4-oxobutyl)(m ethyl)carbamoyl]phenyl}sulfanyl)ethane-1-sulfonic acid (S635), having structure

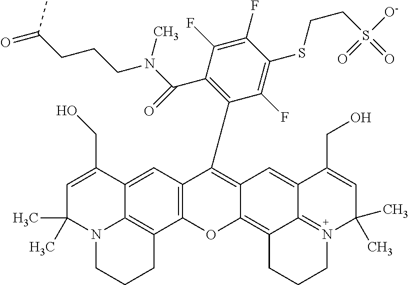

({10,10,22,22-tetramethyl-20-[(phosphonooxy)methyl]-16-{2,3,4,5-tetrafluoro-6-[(4-methoxy-4-oxobutyl)(methyl)carbamoyl]phenyl}-3-oxa-9lambda4,23-diazaheptacyclo[17.7.1.1^{5,9}.0^{2,17}.0^{4,15}.0^{23,27}.0^{13,28}]octacosa-1,4,9(28),11,13,15,17,19(27),20-nonaen-12-yl}methoxy)phosphonic acid (ABBERIOR star 635P), having structure

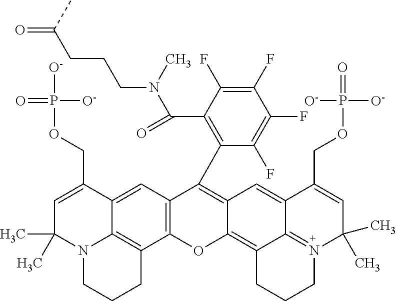

Particularly preferred among these are the groups A chosen in the group consisting of: CF568, AF647, AF568, AF532, Asred, S635 and ABBERIOR star 635P, more preferably between AF647 and AF568.

Next, the method comprises a phase of cytoinclusion of the biological sample subsequent to the supply phase.

The phase of cytoinclusion is performed using a solidifying mixture comprising formalin, paraformaldehyde and at least one organic solvent.

In addition, the phase of preparing the fluorescent donor probe and the phase of preparing the fluorescent acceptor probe comprise at least one step of selecting the Fab fragments or antibodies, respectively.

In detail, the antibody is a rabbit polyclonal antibody of IgG2b type or a mouse monoclonal antibody of IgG1 type or the Fab fragment is derived from an antibody selected from a rabbit polyclonal antibody of IgG2b type or a mouse monoclonal antibody of IgG1 type.

In detail, the Fab fragment or antibody of the acceptor probe and the Fab fragment or antibody of the donor probe are selected from the list comprising: clone #501, clone 3HCLC, clone 42705.111, clone PA5-87555, clone PA5-35089, antiserum PA1-1000.

In addition, the method comprises a phase of de-paraffination of the biological sample treated according to the phase of cytoinclusion, wherein the de-paraffination phase is prior to the phase of setting in contact.

In detail, the fluorescence microspying techniques, preferably super-resolution confocal fluorescence microscopy, comprise FRET microscopy.

In this regard, it should be pointed out that a number of experiments were carried out to validate the resolution of the technical problem mentioned above. The biological sample analyzed consists of a liquid biopsy made up of an aliquot of human venous sampling. The experiments conducted allowed comparing the results obtained by isolating neutrophils from the blood sample with the results obtained by performing the assay on the intact blood aliquot.

### Example 1

### Biological Sample Preparation:

### 1) Sample Preparation for the Immunofluorescence Test:

Take 300 ml of a venous blood sample obtained in the presence of anticoagulant (preferably sodium citrate) and mix it rapidly with the same volume (300 ml) of 8% PFA;
incubate at room temperature for 10 min with occasional shaking;
centrifuge at 400 g for 5 min;
eliminate the supernatant and re-suspend the cell pellet in 1 ml of PBS at room temperature;
vortex the sample until the pellet is completely re-suspended;
centrifuge at 400 g for 5 min;
repeat steps 4-7 (3 times);
cytoinclusion/de-paraffination (Optional, see examples 2 and 3);
wash the deparaffinized cell pellet sections with deionized water or PBS;
re-suspend the pellet obtained from the last step in 250 ml of blocking solution (50 mM NH4Cl, 0.05% P/V saponin, 0.1% P/V BSA in PBS);
incubate 30 min RT;
centrifuge at 400×g for 5 min;
discard the supernatant and re-suspend the cell pellet in 10 ml of blocking solution containing the dilution of the two antibodies (10-50 mg/ml is saturated for both targets);
incubate at 4° C. overnight;
centrifuge at 400×g for 3 min;
eliminate the supernatant and re-suspend the cell pellet in 250 ml PBS
repeat steps 13-14 (3 times);
re-suspend the pellet obtained from the last step in 25 ml of mounting medium (Mowiol 4-88);
deposit the cell suspension on a slide and mount it with a coverslip (0.17 mm for confocal microscope);
let dry 16 to 24 hours before microscopic observation.

### Example 2

### Cytoinclusion

add 5 ml of fixative (10% neutral buffered formalin, 4% paraformaldehyde, 70% ethanol) to the cell pellet and shake lightly. Fix overnight (18-24 hours);
centrifuge at 1500 rpm for 10 minutes at room temperature;
let the supernatant decant;
add 5 ml of 70% ethanol for 30 min and shake lightly on vortex;
centrifuge at 1500 rpm for 10 min at room temperature.
let the supernatant decant;
add 5 ml of 100% ethanol for 30 min and shake lightly on vortex. Optional: overnight incubation in 100% ethanol at 4° C. makes a very solid pellet;
centrifuge at 1500 rpm for 10 min;
let the supernatant decant;
prepare Histogel according to the manufacturer's directions;
add 3-5 ml of liquid Histogel using a clean pipette.
agitate lightly on vortex to distribute and place on ice;
using a clean wooden applicator stick, carefully slide the cell pellet out of the tube and into a cassette lined with black biopsy filter paper. Place the cassette with the pellet in 70% ethanol and submit to histology for processing;
program the tissue processor for a short program (e.g., Program for small samples such as biopsies);
process pellet samples and incorporate them into paraffin within 24 hours from preparation;
cut the cell block into 1 μm sections and induce adhesion on microscope slides.

### Example 3

### De-Paraffination

cell block sections are placed in a jar containing 60 mL of Histolemon for 15 min;
cell block sections are placed in a jar containing 60 mL of Histolemon for min;
gently move the slices from the Histolemon into another jar containing 100% ethanol for 7 minutes;
gently move the sections from 100% ethanol to another jar containing 100% ethanol for 7 minutes;
gently move the sections from 100% ethanol to another jar containing 95% ethanol for 5 minutes;
gently move the sections from 95% ethanol to another jar containing 75% ethanol for 5 minutes;
gently move the sections from 75% ethanol to another jar containing 50% ethanol for 5 minutes;
move the cell block sections in PBS 1× for at least 5 min.

Next, the measured FRET efficiency is related to the abundance of the interaction between the donor and acceptor probe within each pixel of the image. Detection of the FRET signal consists of a precise estimate of the fluorescent emission (FD) intensity or emission decay rate (<τ>) of the donor probe. The quantization of FD in a biological sample appears to be inexpensive in terms of complexity and cost of the instrumentation required for analysis. Calculation of FRET efficiency by estimating FD (acceptor-photobleaching) demonstrated sufficient analytical sensitivity, signal-to-noise ratio and linearity range for evaluation of the receptor interaction under investigation in neutrophil cells. In addition, the developed assay showed high reproducibility among experiments conducted on venous samples taken on different days and coming from different donors. Below is the formula used to calculate the FRET (pixel by pixel) efficiency shown in the images reported in this study:

$${FRET}_{eff} = \frac{{{F}_{D}}_{( post )} - {{F}_{D}}_{( pre )}}{{{F}_{D}}_{( post )}}$$

$${{F}_{D}}_{( PRE )} = donor fluorescence \underline{BEFORE} acceptor bleaching$$

$${{F}_{D}}_{( POST )} = donor fluorescence \underline{AFTER} acceptor bleaching$$

Measuring FRET efficiency by means of FD cannot provide information related to the molecular composition of the complex, such as the ratio of activated (bound) to non-activated (unbound) receptor concentration. In contrast, the measurement of FRET efficiency by <τ> allows this information to be obtained, as the molar fraction of the receptor that does not participate in the multiprotein complex can be extrapolated from the value of <τ> that is obtained. The fitting model used is bi-exponential and the measurement is repeated for each individual neutrophil: a global decay curve consisting of the summation of the curves recorded in all the pixels that make up the analyzed cell is generated for each of them. The rate of decay of the fluorescence intensity of the total donor population (bound and unbound receptor) is of the exponential type and follows the trend described by the formula:

$$F ( t ) = {a}_{1} {e}^{- t / {τ}_{1}} + {a}_{2} {e}^{- t / {τ}_{2}}$$

where τ n identifies the population subject to FRET (bound or unbound receptor), the pre-exponential factor an quantifies its relative abundance corresponding to the mole fraction.

The decay acquired in each region of interest (ROI) corresponds to the lifetime obtained from the cumulative histogram consisting of the summation of all pixels contained in the ROL The histogram is interpolated by bi-exponential fitting, and the lifetime calculated on the two components (A1 and A2) with the following equation:

$$< {τ}_{D} >= {\sum }_{i = 2}^{2} \frac{Ai * ti}{Ai} \qquad ( eq . 1 )$$

with which the intensity weighted lifetime value is calculated in the presence of multi-exponential decay. The values shown in the graphs in this study correspond to the measurements obtained in each individual cell, and the number of samplings is more than adequate for the variance observed for each measurement.

The expression used to calculate the FRET efficiency by using the FLIM technique is as follows:

$$Eff = [ 1 - {t}_{DA} / {t}_{D} ) ] * 100 \% ,$$

where tD is the absolute donor lifetime, measured in a sample containing only the donor probe, and tD-A is the donor lifetime in a sample subjected to the same condition, in which both donor and acceptor probes are present.

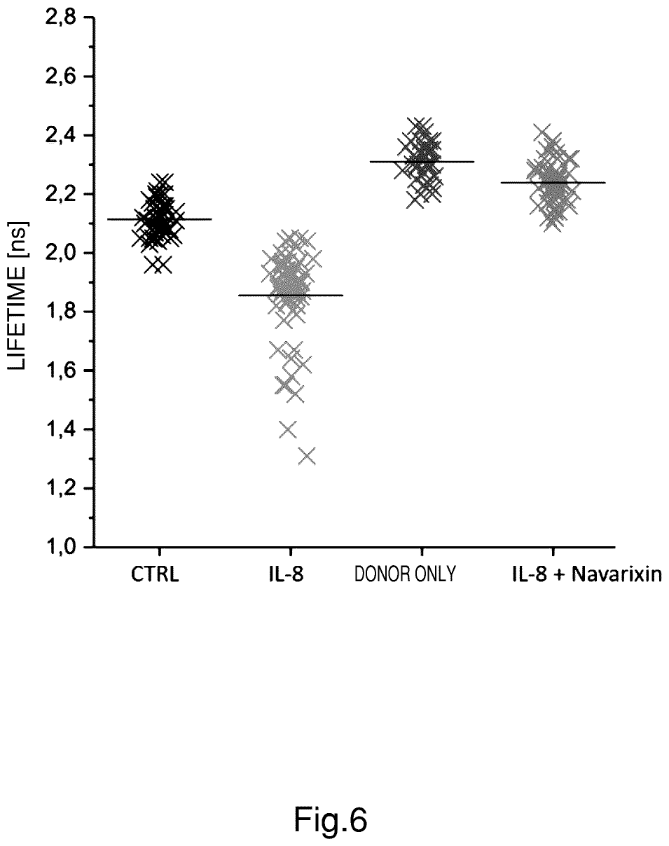

The receptor activation status was measured through the quantification of CXCR1-GaI and CXCR1-Arrestin interactions. Regarding the first pair of interactors, analysis of the results of FRET-FLTIM experiments (FIG. 6) shows the decrease in signal lifetime (indicative of increased donor-acceptor interaction) when in the presence of IL-8, compared with the non-activated control system (in black). Confirmation of the significance of the data is provided by analyzing the system with only the presence of the donor (the anti-CXCR1 antibody), in the absence of the acceptor (anti-GaI). In this circumstance, the minimum value of FRET is assumed, which serves as an internal control. These results confirm the soundness of the detection methodology, despite the fact that the type of GPCR-G protein interaction is known to be rapid, transient and not subject to any kind of accumulation.

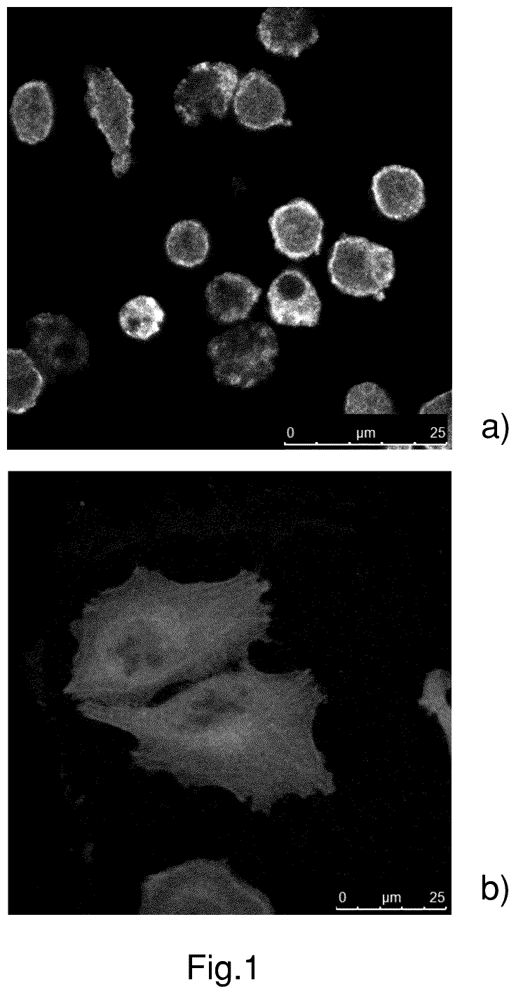

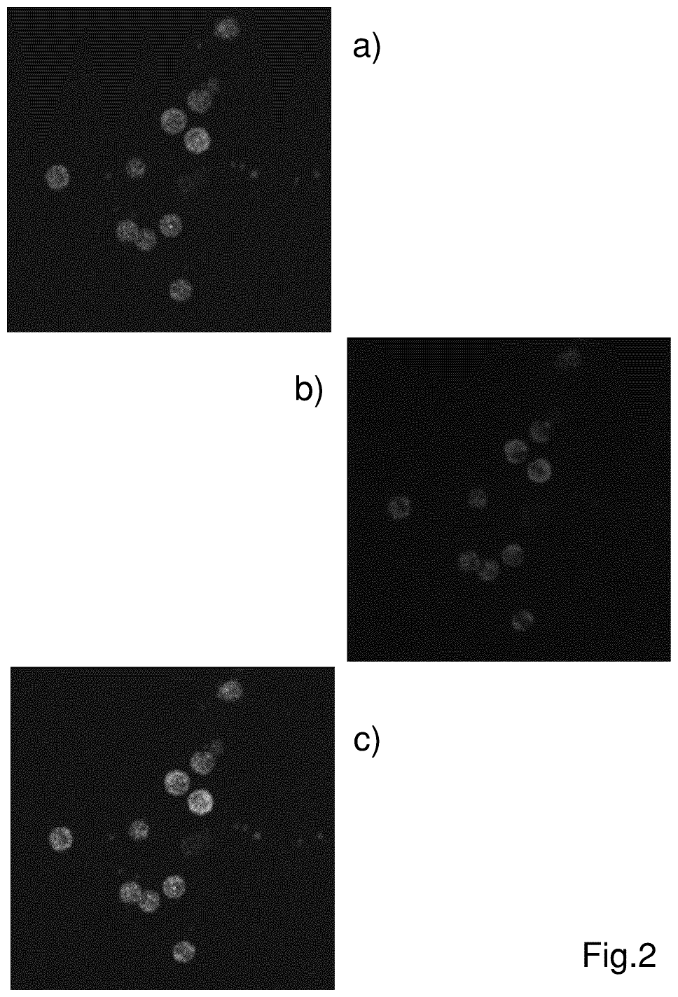

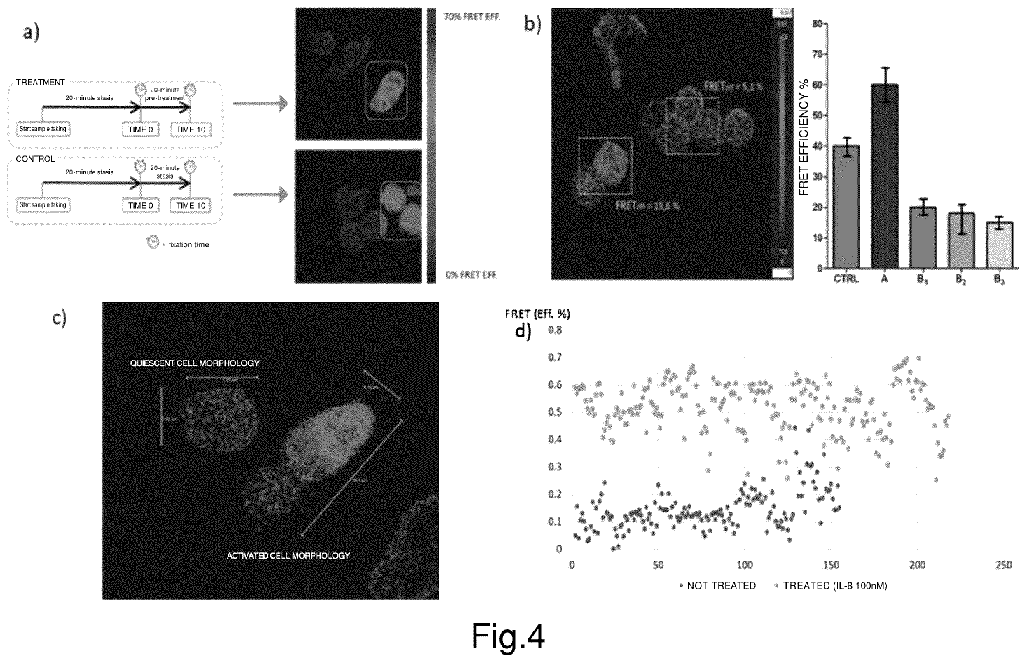

Precisely this last aspect, however, offers added value in monitoring the interaction of the receptor with β-Arrestin. Indeed, in this scenario, the maximum interaction value between these two proteins is recorded 8 to 12 minutes after the interleukin stimulus. The measurement of the activation state occurs within a defined time window, the beginning of which coincides with the time of sampling (FIG. 1a). By subjecting the blood aliquots to a stasis under controlled conditions, it is possible to obtain a homogeneous activation state among the neutrophils in the control sample, estimated by quantifying the amount of receptor that is bound to β-Arrestin at the time of fixation. This measurement allows assessment of the increase in interaction following an IL-8 stimulus, in the presence or absence of allosteric modulator (reparixin). The amount of activation is obtained by difference with the basal state, which corresponds to the amount of desensitized receptor found after the same time interval in each experiment. The correlation between FRET signal intensity and morphology (FIG. 4c) allows us to assess the effectiveness of the inhibitor: the change in morphology from spherical to polarized in the presence of high FRET, indicates the persistence of a G-protein pathway-mediated signal. In contrast, the presence of high FRET in perfectly spherical cells (indistinguishable from those observed in the control aliquot), is an indication of high desensitization of the receptor by β-Arrestins, and thus the inhibitor is able to promote deactivation of the receptor, with the effect of a drastic reduction in G-protein-mediated signaling. In cases where a level of FRET is observed below that measured in the basal condition (FIG. 4c), there is competitive (non-allosteric) inhibition, as found in treatments with hexa-peptide that competes with the IL-8 binding site by blocking the receptor in an inactive conformation (towards both G-protein-mediated and β-Arrestin-mediated signaling).

In stimulated samples, an increase in FRET signal is observed compared with the value recorded in the non-stimulated (control) sample, in accordance with the increased amount of receptor that is desensitized (bound to Arrestins) as a result of the stimulus. In contrast, under a competitive inhibition condition of the receptor, the recorded FRET signal is lower than the value recorded in the control sample (the receptor in complex with β-Arrestins is less abundant).

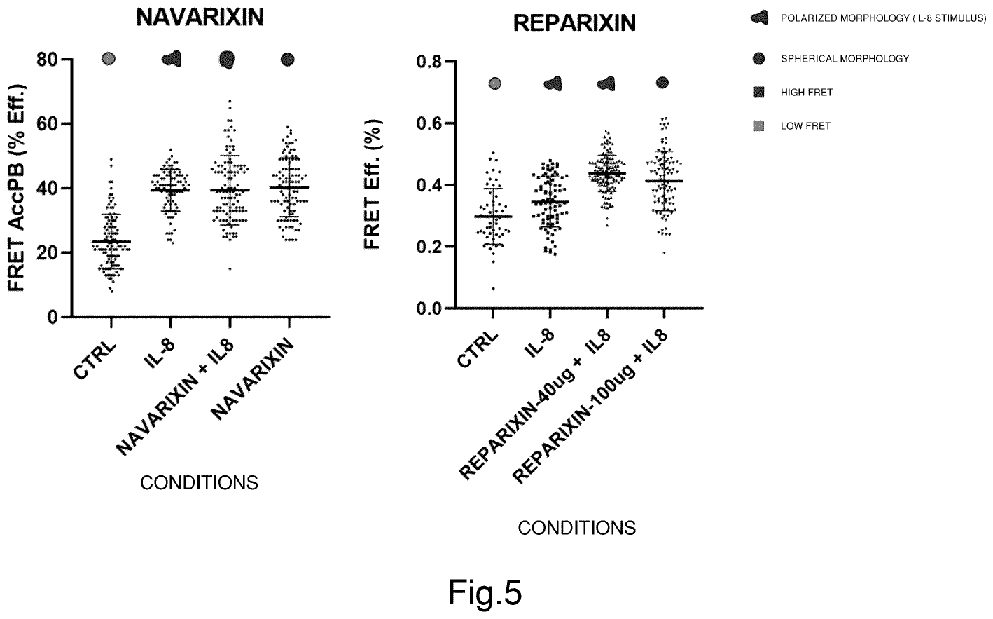

In light of these evaluations, we repeated the same measurement conditions for the analysis of allosteric inhibitors (Navarixin and Reparixin). In samples subjected to combined treatments (IL-8+allosteric inhibitor), we observe that FRET is higher than control, indicative of increased interaction with β-Arrestins. Thus, the treatment with allosteric inhibitors generates high receptor desensitization. However, if IL-8 treatment is conducted in the presence of allosteric inhibitor, neutrophil morphology is maintained spherical even under stimulated conditions. This result is in perfect analogy with the observations conducted in the quiescent or competitive inhibition state (competitive peptide treatments). In the quiescent or competitive inhibition condition, the neutrophil consistently retains a spherical morphology. The results obtained under the various conditions are shown in FIG. 5.

The most striking difference between the condition in which the sample is treated with IL-8 and the condition in which the sample is pretreated with inhibitor and then subjected to the IL-8 stimulus concerns morphology. The FRET signal is similar, but this, upon fixation, is polarized in the samples in which the inhibitor was absent and perfectly spherical in the samples pre-treated with the inhibitor. In actual facts, in cells treated with the inhibitor, the FRET signal between CXCR1-betaARRESTIN1 reaches a value close to that obtained by doing the treatment with only IL-8 (FIG. 5).

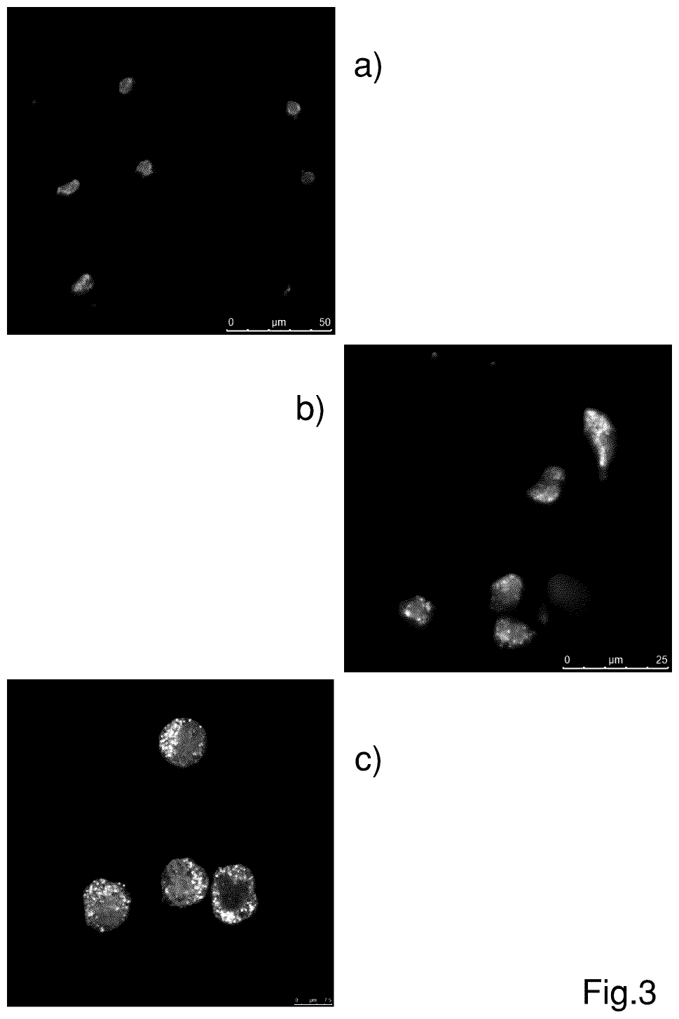

Modulation of CXCR1-GaI interaction also reflects the activity of allosteric modulator treatment, in this case NAVARIXIN. Indeed, as shown in FIG. 3, the decay time of the FRET signal in the presence of the inhibitor is significantly longer than that of the activated state, suggesting the unbalancing of the receptor population towards the non-Gi2-bound state.

Experiments are underway with treatments carried out exclusively with REPARIXIN, so as to systematically compare the results obtained with different inhibitors in relation to the quiescent state and the activated state.

Another aspect that strongly distinguishes the sample activated with IL8 alone and the IL8_+_navarixin sample is the cell count after treatment. The sample pretreated with inhibitor and then stimulated with IL-8 has the same number of cells compared to those found at the control, whereas in the sample stimulated with IL-8 alone, the cells found at the time of fixation turn out to be about 20 percent of those in the control, along with a lot of cell debris and half-exploded cells.

Another crucial factor—from the experimental point of view—is the treatment time: the treatment with the allosteric inhibitor must occur at the time of the sampling, and the inhibitor must remain in the aliquot of whole blood during the 20-minute stasis before carrying out the IL-8 treatment. The combined and concerted addition of IL-8 and inhibitor produces the same effect as interleukin alone, thus generating an activation condition absolutely indistinguishable from the samples treated with IL-8 stimulus alone.

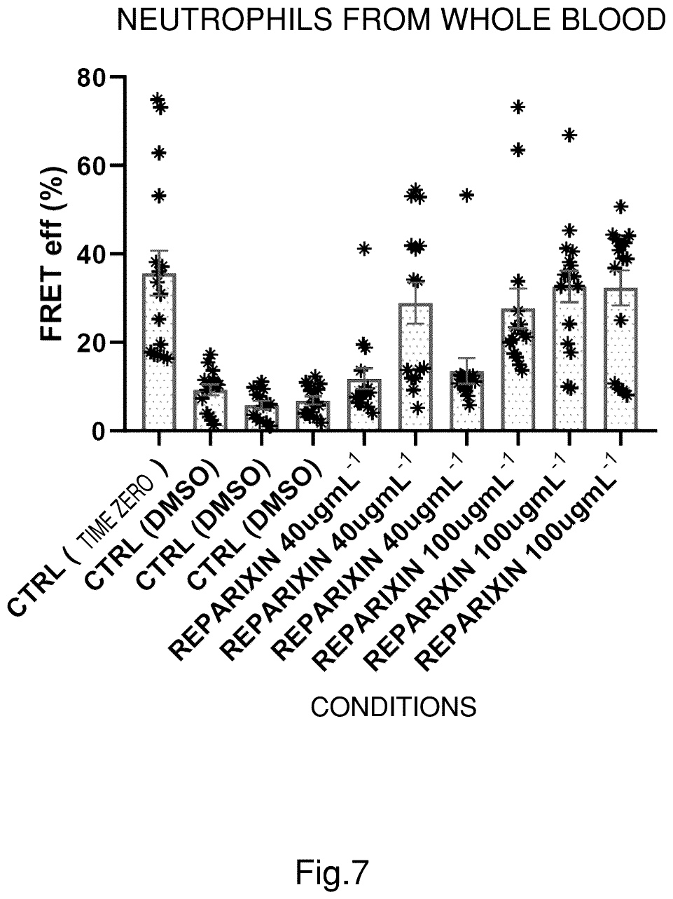

Next, the FRET assay for measuring the CXCR1//ArrB interaction on neutrophils was repeated, operating in triplicate (FIG. 4). A first control sample, CTRL-DMSO, corresponds to the sample set at 30 minutes; this time is required for neutrophils to return to a resting state after mechanical activation due to handling. To confirm this, a second control sample, CTRL-time zero, set after only 2 minutes, was considered. The treatments, on the other hand, involve the addition of Reparixin at the two concentrations of interest, 40 and 100 μgmL-1. Values reported refer to the measurements on 15 neutrophil cells of donor fluorophore de-quenching following acceptor photo-bleaching, obtained by the INTENSITY-BASED ACCEPTOR PHOTOBLEACHING approach (FIG. 7 and Table 1).

TABLE 1

| | CTRL | | | | | | | | | |
|---|---|---|---|---|---|---|---|---|---|---|
| | upon | CTRL | | | REPARIXIN | | | REPARIXIN | | |
| | sampling | (DMSO) | | | 40 ugmL [symbol] | | | 100 ugmL [symbol] | | |
| Median | 32.28 | 10.50 | 6.000 | 6.480 | 8.770 | 33.92 | 11.23 | 21.92 | 35.03 | 38.96 |
| Mean | 35.63 | 9.269 | 5.822 | 6.853 | 11.80 | 28.90 | 13.52 | 27.67 | 32.60 | 32.35 |
| Std. | 20.19 | 4.733 | 3.305 | 3.439 | 9.260 | 18.03 | 11.16 | 17.35 | 14.24 | 15.30 |
| Deviation | | | | | | | | | | |
| Std. Error | 5.048 | 1.222 | 0.9166 | 0.8878 | 2.391 | 4.654 | 2.861 | 4.480 | 3.560 | 3.951 |
| of Mean | | | | | | | | | | |

[symbol]

indicates data missing or illegible when filed

FRET is high in many cells analyzed in the sample treated at the highest concentration of Reparixin, while in two of the samples treated with Reparixin at a dose of 40 ug/ml many cells with FRET values equal to the control (CTRL) are found. The CTRL of each triplicate consists of a sister sample subjected to the same treatment conditions unless drug addition. Exclusively for the time zero sample, fixation was carried out at about one minute after sampling, and the analysis reported high FRET probably due to heterologous desensitization. For all remaining samples, fixation was performed at 30 minutes after sampling, an interval of time during which the drug was allowed to act, directly added to the tube of each treated sample.

With the aim of confirming the results obtained with the assay just described, a second round of experiments was conducted, this time aimed at determining the lifetime measurement of the fluorescence emitted from the donor probe (carried by the anti-CXCR1 antibody) to the acceptor probe (on the anti-βArrestin2 antibody), to support the interpretation of the results of the FRET experiments. In fact, this measurement is usually coupled to the previous one to provide a clear picture of the interaction events between the proteins of interest taking place in the sample. The fluorophores are the same as in the previous experiment; the donor probe is regio-selectively labeled with a single fluorophore molecule, while the acceptor probe is labeled with 6 fluorophore molecules, to realize the system described in our papers (Antenna System: J Phys Chem B, 115(33):10120-5; J Biomed Opt.; 17(1):011006).

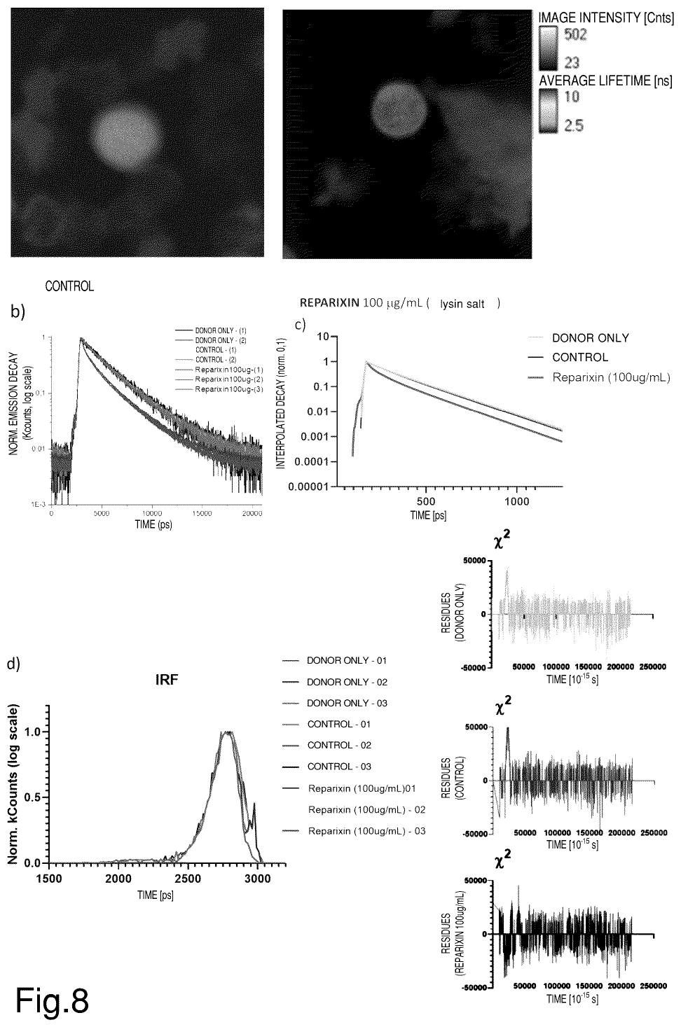

The fluorescent emission decay of the donor probe under the 3 different conditions is shown below: CTRL (control, corresponding to quiescent cells), Donor Only (internal negative control in which the acceptor probe is missing), Reparixin 100 micrograms/mL, which corresponds to the highest tested assay. To correctly interpret the following graph (FIG. 8b), remember that the presence of interaction induces a lowering of the donor probe lifetime; this lowering is quantified by the slope of the decay curve, which corresponds to the donor probe lifetime (τ). The value of τ can be derived numerically from the nonlinear fitting of the decay curve. The method of FRET analysis by lifetime measurement represents the state of the art for energy transfer measurements, as it consists of a spectroscopic measurement, the magnitude of which is independent of both fluctuations in laser source intensity and of the expression level of the analyzed proteins and other concentration-dependent effects.

It is evident how the basal interaction level found in the “CTRL” neutrophil sample, i.e., control (corresponding to the quiescent state), is very slightly higher than that found in samples containing the “donor” probe only, in which FRET is absent. All this can be seen independently of the fitting model (described below), and the “raw” data (i.e., the non-fitted decay curves) are perfectly consistent with the results obtained by the “Acceptor Photobleaching” method conducted on the samples prepared in the context of the same experiment. It is very interesting to note that the increase in the decay rate of fluorescent emission is significant and thus can be visually appreciated from the recorded curves, without the need to perform nonlinear fitting for determining the precise value of τ (NLLS analysis). Each curve represents the raw data of the FLIM analysis, obtained from the summation of the emission decay curves recorded in the region of interest (ROI), which corresponds to an area including each neutrophil that is therefore analyzed individually. An alternative analysis method consists of pixel-by-pixel representation of the average arrival time of each photon collected in the confocal frame scan (average photon arrival time, APAT). The collected images related to the APAT are shown below (FIG. 8a), and allow assessing the morphology of the interaction: as can be seen, the detected interaction has a predominantly intracellular localization, which is compatible with the increased internalization of the receptor.

For greater precision, we carried out further analysis of the recorded curves by verifying that for each of them the instrumental response function (IRF) was superimposable. The IRF of each measurement makes it possible to quantify the presence of non-ideal behavior in the measurement, due to both instrumental and sample-dependent factors, for each acquisition: thus, quantitative comparison of the decay curves obtained in separate acquisitions is possible.

IRF is the instrumental response function which comprises the “dead time” of the phototube used (the electronics in our PMT have a dead time of about 200 picoseconds, but this value is subject to change depending on the aging of the photosensitive material and the wavelength used). In addition, IRF also comprises the delay due to the time width of the excitation pulse, which under real conditions is not point-like (like a pure dirac), but actually consists of a train of pulses for a total time width of about 200 picoseconds. For a proper comparison of the different decay curves acquired in independent slides, also in order to obtain an accurate value of r for each of them, it is important to record IRF for each decay and to perform a convolution of the IRF function from the obtained histogram (i.e., the non-fitted curve shown in FIG. 8) by accumulating the values of photon arrival time for each digital time bins (corresponding to the abscissa values in the curves in the first figure). The IRFs for each recorded decay (≈250 ps) are shown in FIG. 8, panel (d).

As can be seen from the graph, the IRF of each measurement has an FWHM that is superimposable on the others, so it is possible to compare the curves recorded under the various treatment conditions, and then proceed to nonlinear fitting to calculate the lifetime value from each cell. The fitted curves for 3 cells are shown in Figure FIG. 8, panel (c). The Levenberg-Marquardt algorithm, which is based on the method of least squares, was used for nonlinear fitting of the recorded decay curves. Therefore, in order to compare the values of τ calculated from the different curves, the trend of X² of each measurement throughout the acquisition range (from 0 to 25 ns) is plotted in a graph, to assess whether the mean deviation between the fitted values (shown in the last figure) and the experimental values of the histogram (those in the first figure) is consistent with the measurements obtained from independent slides (i.e., the various treatment conditions), so that the results obtained from the fitting (the different values of τ) can be compared in a sufficiently rigorous manner. In FIG. 8, panel (e), the trend of the mean square deviation (2) is shown for the three fitted curves according to a tri-exponential model. This value approximates unity, so the estimate of lifetime values obtained in this set of measurements is very accurate.

As can be seen, the mean square deviation X² observed between the values produced by the exponential fitting and those experimentally obtained from the recorded decay is highly coincident among the three measurements made, particularly between 5 nanoseconds and 10 nanoseconds, which is the area of the decay where the emissive component with rapid decay due to the presence of energy transfer is most visible. The values of r extrapolated from the nonlinear fitting by using the Levenberg-Marquardt algorithm were further verified using the maximum likelihood estimation (MLE) method, which allows for greater accuracy in evaluating the parameters obtained from the statistical distribution of photonic phenomena (such as the decay of a fluorophore's emission), as these phenomena are generally governed by a Poissonian distribution. The values shown in the table are those calculated exclusively by the Levenberg-Marquardt algorithm.

The table below shows the values found in the analysis of 3 cells for each condition:

| | | | | | Variability | | | |
|---|---|---|---|---|---|---|---|---|
| | | | | Std. | Index | | | |
| | | | | Error | (mean | | | |
| | | | | of | simple | | | |
| CONDITION | τ | | | Mean | deviation) | χ² | | |
| Control | 2.60 | 2.57 | 2.63 | 0.1732 | 0.04 | 1.03 | 1.47 | 1.35 |
| Donor only | 2.66 | 2.67 | 2.64 | 0.08819 | 0.13 | 1.22 | 1.08 | 1.13 |
| Reparixin | 2.05 | 2.12 | 2.1 | 0.02082 | 0.03 | 1.35 | 1.24 | 1.07 |
| 100 μg/ ml | | | | | | | | |

The reported data show that IL-8 estimation produces a significant increase in the amount of receptor in the desensitized state (and thus in complex with b-Arrestin2), both in the presence and absence of allosteric modulator. This aspect is decisive in defining the effect of allosteric modulation of the CXCR1 receptor: allosteric inhibition capable of inhibiting IL-8-mediated receptor activation thus consists of 1) the lack of activation of the G-protein-mediated pathway (spherical morphology) and 2) the consequent increase in Arrestin-mediated signal (high FRET). Indeed, in the absence of G-protein-triggered signal, neutrophil migration is inhibited and the morphology remains spherical, as reported in conventional chemotaxis experiments reported in the literature.

In conclusion, it has been verified that in quiescent samples, held under optimal conditions capable of strongly minimizing the existing basal receptor-arrestin interaction (i.e., heterologous desensitization due to mechanical stress or involuntary pre-activation, which occurs, for example, during Ficoll gradient isolation), FRET is almost zero. Under treatment conditions, if properly conducted, there is an increase in FRET around 30-40%, which is a considerable value. Possible peaks up to 70% are also possible, but have not yet been verified by FLIM technique. In practice, it is possible that treatment at different dose (40 μg/mL or 100 μg/mL) causes an increase in the number of high FRET cells rather than a different absolute value of FRET.

1) A method for measurement of an activation state of G-protein-coupled receptors, the method comprising:
supplying a human biological sample;
preparing at least one fluorescent donor probe comprising an anti-GPCR Fab fragment or antibody and a fluorophore group linked via amide bond to an amino group of a N-terminal amino acid of a light chain and/or of a N-terminal amino acid of a heavy chain of said Fab fragment or antibody;
preparing at least one fluorescent acceptor probe comprising an anti-G-protein and/or anti-arrestin Fab fragment or antibody and a fluorophore group linked via amide bond to an amino group of the N-terminal amino acid of the light chain and/or of the N-terminal amino acid of the heavy chain of said Fab fragment or antibody;
setting said human biological sample in contact with said fluorescent acceptor probe and with said fluorescent donor probe; and
sensing the activation state of said GCPR receptors by means of fluorescence microscopy techniques.

2) The method according to claim 1, further comprising: a phase of cytoinclusion of said biological sample subsequent to said supply phase.

3) The method according to claim 2, wherein said phase of cytoinclusion is carried out by means of a solidifying mixture comprising formalin, paraformaldehyde and at least one organic solvent.

4) The method according to claim 1, wherein said phase of preparing said fluorescent donor probe and said phase of preparing said fluorescent acceptor probe comprise at least one step of selecting said Fab fragments or antibodies, respectively.

5) The method according to claim 1, wherein said antibody is a rabbit polyclonal antibody of the IgG2b type or a mouse monoclonal antibody of the IgG1 type or said Fab fragment is derived from an antibody selected from a rabbit polyclonal antibody of the IgG2b type, or a mouse monoclonal antibody of the IgG1 type.

6) The method according to claim 1, wherein said fluorescent donor probe comprises an anti-CXCR1 and anti-CXCR2 Fab fragment or antibody.

7) The method according to claim 1, wherein said fluorescent acceptor probe comprises an anti-sub unit G-α-I and/or anti-ß-arrestin 1 and/or anti-ß-arrestin 2 Fab fragment or antibody.

8) The method according to claim 1, wherein said Fab fragment or antibody of said acceptor probe and said Fab fragment or antibody of said donor probe are selected from the list comprising: clone #501, clone 3HCLC, clone 42705,111, clone PA5-87555, clone PA5-35089, antiserum PA1-1000.

9) The method according to claim 1, further comprising: at least one phase of de-paraffination of said biological sample treated according to said phase of cytoinclusion, said de-paraffination phase being prior to said phase of setting in contact.

10) The method according to claim 1, wherein said biological sample is obtained from a patient treated with a pharmacological modulator of interleukin-8 receptors.
